(19) United States
(12) Patent Application Publication
(10) Pub. No.: US 2022/0392009 A1
(43) Pub. Date: Dec. 8, 2022

(54) Method of Performing Mobility Sharing Based on Disinfection Information and Apparatus for the Same

(71) Applicants: Hyundai Motor Company, Seoul (KR); Kia Corporation, Seoul (KR)

(72) Inventors: Jae Jun Ha, Hwaseong-si (KR); Young Jun Moon, Sejong-si (KR); Tae Hee Lee, Yongin-si (KR)

(21) Appl. No.: 17/726,872

(22) Filed: Apr. 22, 2022

(30) Foreign Application Priority Data
Jun. 4, 2021 (KR) 10-2021-0072796

(51) Int. Cl. G06Q 50/30; G16H 50/30

(52) U.S. Cl. CPC G06Q 50/30; G16H 50/30

(57) ABSTRACT

An embodiment method of providing a mobility sharing service based on disinfection information includes receiving, at a mobility sharing management server, a mobility sharing service request from a user terminal, requesting, by the mobility sharing management server, disinfection information from a disinfection management server based on location information of the user terminal, receiving, by the mobility sharing management server, the disinfection information from the disinfection management server and calculating a predicted infection degree based on the disinfection information, and determining whether to provide the mobility sharing service to a user corresponding to the user terminal based on the predicted infection degree.

### CROSS-REFERENCE TO RELATED APPLICATIONS

This application claims the benefit of Korean Patent Application No. 10-2021-0072796, filed on Jun. 4, 2021, which application is hereby incorporated herein by reference.

### TECHNICAL FIELD

The present disclosure relates to a method and apparatus for providing a mobility sharing service based on disinfection information.

### BACKGROUND

As technology advances, the concept of vehicle ownership has been weakened and use of shared vehicles has increased. Specifically, although the need to share vehicles such as car sharing or carpooling is increasing, the existing shared vehicles do not reflect diverse tendencies of individual users. Therefore, a personal interaction system of a user may be required.

In addition, vehicles are shared in a specific area or residence for convenience of life, and the need to receive services using them is increasing. However, in the case of sharing a vehicle, satisfaction with the service is not high compared to a privately owned vehicle. Since a vehicle sharing system is not properly established, it may be difficult to provide vehicle security and service. Hereinafter, a vehicle fleet system will be provided in consideration of the foregoing.

Furthermore, the spread of infectious diseases through viruses is emerging as a social problem. In this situation, use of a mobility sharing service is avoided. In order to solve this, it is required to provide a mobility sharing service capable of preventing the spread of infectious diseases.

### SUMMARY

Embodiments of the present disclosure may provide a method and apparatus for providing a mobility sharing service capable of preventing the spread of infectious diseases using disinfection information.

According to an embodiment of the present disclosure, provided is a method of providing a mobility sharing service based on disinfection information. The method of providing the mobility sharing service based on disinfection information may include a user terminal transmitting a mobility sharing service request to a mobility sharing management server, the mobility sharing management server requesting disinfection information from a disinfection management server based on location information of the user terminal, the mobility sharing management server receiving the disinfection information from the disinfection management server and calculating a predicted infection degree based on the disinfection information, and determining whether to provide the mobility sharing service to a user corresponding to the user terminal using the predicted infection degree.

The calculating the predicted infection degree may include checking at least one of environment factors corresponding to a mobility sharing service area or personal factors corresponding to the user, and calculating the predicted infection degree based on at least one of the environment factors or the personal factors.

The environment factors may include at least one of information on population density of the mobility sharing service area, information on a rate of confirmed cases in an area, information on a rate of confirmed cases according to age or information on an increase in the number of confirmed cases.

The checking at least one of the environment factors or the personal factors may include checking at least one essential element included in the personal factors and checking whether the at least one essential element exceeds a predetermined threshold.

The checking at least one of the environment factors or the personal factors may include checking at least one general element included in the personal factors as the at least essential element exceeds a predetermined threshold.

The checking at least one of the environment factors or the personal factors may include checking at least one of an essential element or a general element included in the personal factors and determining the personal factors using at least one of the checked essential element or general element.

The at least one essential element may include contact risk information numerically indicating a user's interpersonal contact tendency.

The at least one general element may include at least one of a user's activity level numerically indicating a user's activity tendency or an immunity index numerically indicating an ability to defend against a disease of the user.

The method of providing the mobility sharing service based on disinfection information may further include a mobility transmitting a mobility return request to a mobility sharing management server, the mobility sharing management server requesting disinfection information from a disinfection management server based on the location information of the mobility, the mobility sharing management server receiving the disinfection information from the disinfection management server, calculating the predicted infection degree of the mobility based on the disinfection information, and determining whether to reshare the mobility using the predicted infection degree of the mobility.

The determining whether to reshare the mobility may include determining whether to reshare the mobility by comparing the predicted infection degree of the mobility with a predetermined threshold.

The determining whether to reshare the mobility may include determining a disinfection process for the mobility in response to the predicted infection degree of the mobility exceeding the predetermined threshold.

The determining the disinfection process for the mobility may include determining a destination of the mobility to be a disinfection area.

The method may further include requesting movement to the disinfection area.

The method may further include controlling movement to the disinfection area.

The determining whether to reshare the mobility may include determining that a subsequent user reshares the mobility in response to the predicted infection degree of the mobility being equal to or relatively smaller than the predetermined threshold.

The method may further include determining a destination of the mobility to be a return area.

The method may further include requesting movement to the return area.

The method may further include controlling movement to the return area.

The calculating the predicted infection degree of the mobility may include checking environment factors corresponding to the location information of the mobility and calculating the predicted infection degree of the mobility based on the environment factors.

The method of providing the mobility sharing service based on the disinfection information may further include a mobility transmitting a mobility state check request to a mobility sharing management server, the mobility sharing management server requesting disinfection information from a disinfection management server based on the location information of the mobility, the mobility sharing management server receiving the disinfection information from the disinfection management server, calculating a predicted infection degree of the mobility based on the disinfection information, and determining whether to maintain use of the mobility.

The determining whether to maintain use of the mobility may include determining whether to maintain use of the mobility by comparing the predicted infection degree of the mobility with a predetermined threshold.

The determining whether to maintain use of the mobility may include determining a disinfection process of the mobility in response to the predicted infection degree of the mobility exceeding the predetermined threshold.

The determining the disinfection process for the mobility may include determining a destination of the mobility to be a disinfection area.

The method may further include requesting movement to the disinfection area.

The method may further include controlling movement to the disinfection area.

The determining whether to maintain use of the mobility may include determining that use of the mobility is maintained, in response to the predicted infection degree of the mobility being equal to or relatively smaller than the predetermined threshold.

The method may further include the mobility sharing management server checking a potential risk of the user.

The checking the potential risk may further include checking a main waypoint or a main destination based on a user's history for use of the mobility sharing service.

The checking the potential risk may further include checking confirmed case information corresponding to the main waypoint or the main destination.

The checking the potential risk may further include guiding avoidance of the main waypoint or the main destination in consideration of the confirmed case information.

The checking the potential risk may further include determining whether the mobility sharing service is available in consideration of the confirmed case information corresponding to the main waypoint or the main destination.

According to another embodiment of the present disclosure, a system for providing a mobility sharing service may be provided. The system for providing the mobility sharing service based on disinfection information may include a mobility, a user terminal configured to request a mobility sharing service of a user and to receive and provide whether to provide the mobility sharing service, and a mobility sharing management server configured to request disinfection information from a disinfection management server based on location information of the user terminal and to determine whether to provide the mobility sharing service to a user corresponding to the user terminal based on the disinfection information received from the disinfection management server.

According to another embodiment of the present disclosure, a method of providing a mobility sharing service by a mobility may be provided. The method may include approving use of the mobility sharing service of a mobility user, checking location information of the mobility while the user uses the mobility sharing service, providing the location information of the mobility to a mobility sharing management server, and receiving whether to reshare the mobility from the mobility sharing management server. Here, whether to reshare the mobility may be determined using a predicted infection degree calculated based on the location information of the mobility.

Whether to reshare the mobility may include information indicating whether a disinfection process of the mobility is required.

The method may include performing notice of movement of the mobility to a disinfection area as the disinfection process of the mobility is required.

The method may include setting a destination of the mobility to a disinfection area as the disinfection process of the mobility is required.

According to another embodiment of the present disclosure, a mobility for providing a mobility sharing service may be provided. The mobility may include at least one transceiver configured to transmit and receive a signal, and at least one processor configured to control the at least one transceiver. The at least one processor may approve use of the mobility sharing service by a user based on assignment information received from a mobility sharing management server and use request information received from a user terminal corresponding to the user, check location information of the mobility while the user uses the mobility sharing service, and receive whether to reshare the mobility from the mobility sharing management server. Whether to reshare the mobility may be determined using a predicted infection degree calculated based on the location information of the mobility.

According to another embodiment of the present disclosure, a method of providing a mobility sharing service by a server apparatus may be provided. The method may include receiving a mobility sharing service request from a user terminal, requesting disinfection information from a disinfection management server based on location information of the user terminal, receiving the disinfection information from the disinfection management server and calculating a predicted infection degree based on the disinfection information, and determining whether to provide the mobility sharing service to a user corresponding to the user terminal using the predicted infection degree.

The calculating the predicted infection degree may include checking at least one of environment factors corresponding to a mobility sharing service area or personal factors corresponding to the user, and calculating the predicted infection degree based on at least one of the environment factors or the personal factors.

The checking at least one of the environment factors or the personal factors may include checking at least one essential element included in the personal factors and checking whether the at least one essential element exceeds a predetermined threshold.

The checking at least one of the environment factors or the personal factors may include checking at least one general element included in the personal factors as the at least essential element exceeds a predetermined threshold.

The checking at least one of the environment factors or the personal factors may include checking at least one of an essential element or a general element included in the personal factors and determining the personal factors using at least one of the checked essential element or general element.

The method may further include receiving a mobility return request from the mobility, requesting disinfection information from a disinfection management server based on location information of the mobility, receiving the disinfection information from the disinfection management server and calculating a predicted infection degree of the mobility based on the disinfection information, and determining whether to reshare the mobility using the predicted infection degree of the mobility.

The determining whether to reshare the mobility may include determining whether to reshare the mobility by comparing the predicted infection degree of the mobility with a predetermined threshold.

The determining whether to reshare the mobility may include determining a disinfection process for the mobility in response to the predicted infection degree of the mobility exceeding the predetermined threshold.

The determining the disinfection process for the mobility may include determining a destination of the mobility to be a disinfection area.

The method may further include requesting movement to the disinfection area from the mobility.

The determining whether to reshare the mobility may include may include determining that a subsequent user reshares the mobility in response to the predicted infection degree of the mobility being equal to or relatively smaller than the predetermined threshold.

The method may further include determining a destination of the mobility to be a return area.

The method may further include requesting movement to the return area.

The calculating the predicted infection degree of the mobility may include checking environment factors corresponding to the location information of the mobility and calculating the predicted infection degree of the mobility based on the environment factors.

The method may further include the mobility sharing management server checking a potential risk of the user.

According to another embodiment of the present disclosure, a server apparatus for providing a mobility sharing service may be provided. The server apparatus may include at least one transceiver configured to transmit and receive a signal, and at least one processor configured to control the at least one transceiver. The at least one processor may receive a mobility sharing service request from a user terminal, request disinfection information from a disinfection management server based on location information of the user terminal, receive the disinfection information from the disinfection management server, calculate a predicted infection degree based on the disinfection information, and determine whether to provide the mobility sharing service to a user corresponding to the user terminal.

According to embodiments of the present disclosure, it is possible to provide a method of providing a mobility sharing service capable of preventing the spread of infectious diseases using disinfection information.

According to embodiments of the present disclosure, it is possible to provide a mobility sharing service capable of preventing the spread of infectious diseases due to mobility sharing by predicting an infection degree of a user or mobility using disinfection information.

### BRIEF DESCRIPTION OF THE DRAWINGS

FIG. 1 is a view illustrating a method of performing communication between a mobility and another mobility or a device through a network.

FIG. 2A is a view schematically illustrating a mobility sharing service system based on disinfection information according to an embodiment of the present disclosure.

FIG. 2B is a view schematically illustrating a mobility sharing service area and a disinfection area used in a mobility sharing service system based on disinfection information according to an embodiment of the present disclosure.

FIGS. 3A and 3B are signal flow diagrams illustrating operation of a mobility sharing service system based on disinfection information according to an embodiment of the present disclosure.

FIG. 4A is a signal flow diagram illustrating a mobility return operation of a mobility sharing service system based on disinfection information according to an embodiment of the present disclosure.

FIG. 4B is a signal flow diagram illustrating a mobility control operation of a mobility sharing service system based on disinfection information according to an embodiment of the present disclosure.

FIG. 5 is a diagram illustrating a predicted infection level calculation operation used in a mobility sharing service system based on disinfection information according to an embodiment of the present disclosure.

FIG. 6A is a diagram illustrating a contact risk level used in a mobility sharing service system based on disinfection information according to an embodiment of the present disclosure.

FIG. 6B is a diagram illustrating a user's activity used in a mobility sharing service system based on disinfection information according to an embodiment of the present disclosure.

FIG. 6C is a diagram illustrating a user's immunity index used in a mobility sharing service system based on disinfection information according to an embodiment of the present disclosure.

FIG. 7 is a diagram illustrating the configuration of an apparatus according to an embodiment of the present disclosure.

### DETAILED DESCRIPTION OF ILLUSTRATIVE EMBODIMENTS

Hereinafter, exemplary embodiments of the present disclosure will be described in detail with reference to the accompanying drawings so that those skilled in the art may easily implement the present disclosure. However, the present disclosure may be implemented in various different ways, and is not limited to the embodiments described herein.

In describing exemplary embodiments of the present disclosure, well-known functions or constructions will not be described in detail since they may unnecessarily obscure the understanding of the present disclosure. The same constituent elements in the drawings are denoted by the same reference numerals, and a repeated description of the same elements will be omitted.

In the present disclosure, when an element is simply referred to as being “connected to”, “coupled to” or “linked to” another element, this may mean that an element is “directly connected to”, “directly coupled to” or “directly linked to” another element or is connected to, coupled to or linked to another element with yet another element intervening therebetween. In addition, when an element “includes” or “has” another element, this means that one element may further include another element without excluding another component unless specifically stated otherwise.

In the present disclosure, the terms first, second, etc. are only used to distinguish one element from another and do not limit the order or the degree of importance between the elements unless specifically mentioned. Accordingly, a first element in an embodiment could be termed a second element in another embodiment, and, similarly, a second element in an embodiment could be termed a first element in another embodiment, without departing from the scope of the present disclosure.

In the present disclosure, elements that are distinguished from each other are for clearly describing each feature, and do not necessarily mean that the elements are separated. That is, a plurality of elements may be integrated in one hardware or software unit, or one element may be distributed and formed in a plurality of hardware or software units. Therefore, even if not mentioned otherwise, such integrated or distributed embodiments are included in the scope of the present disclosure.

In the present disclosure, elements described in various embodiments do not necessarily mean essential elements, and some of them may be optional elements. Therefore, an embodiment composed of a subset of elements described in an embodiment is also included in the scope of the present disclosure. In addition, embodiments including other elements in addition to the elements described in the various embodiments are also included in the scope of the present disclosure.

The advantages and features of embodiments of the present invention and the way of attaining them will become apparent with reference to exemplary embodiments described below in detail in conjunction with the accompanying drawings. Embodiments, however, may be embodied in many different forms and should not be construed as being limited to the example embodiments set forth herein. Rather, these embodiments are provided so that this disclosure will be complete and will fully convey the scope of the invention to those skilled in the art.

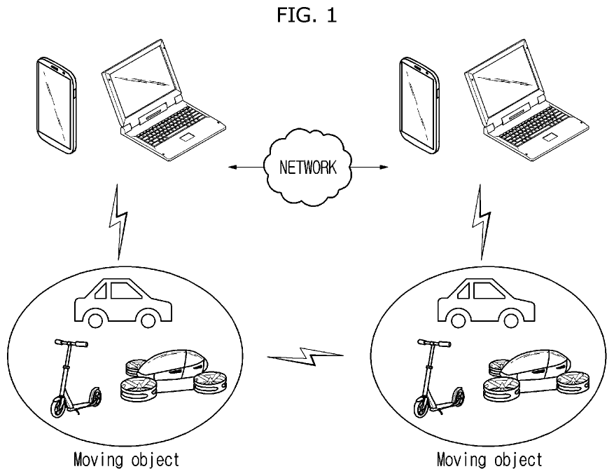

FIG. 1 is a view illustrating a moving object performing communication with another apparatus through a network.

Referring to FIG. 1, the moving object may perform communication with another moving object or another device. For example, the moving object may perform communication with another moving object or another device based on cellular communication, WAVE communication, dedicated short range communication (DSRC) or another communication method. That is, as the cellular communication network, a communication network such as LTE or 5G, a Wi-Fi communication network, a WAVE communication network, etc. may be used. In addition, a short-range communication network used in a moving object, such as DSRC, may be used, but the present disclosure is not limited to the above-described embodiment.

In addition, for example, in relation to communication of the moving object, for security of the moving object, a module capable of performing communication with a device located inside the moving object and a module capable of performing communication with a device located outside the moving object may be separately provided. For example, in the moving object, communication with only a device in a predetermined range in the moving object, such as Wi-Fi communication, may be performed based on security. For example, the moving object and the personal user terminal of the driver of the moving object may include communication modules for performing communication between each other. That is, the moving object and the personal user terminal of the driver of the moving object may use a communication network blocked from an external communication network. In addition, for example, the moving object may include a communication module for performing communication with an external device. In addition, for example, the above-described modules may be implemented as one module. That is, based on one module, the moving object may perform communication with another device, but the present disclosure is not limited to the above-described embodiments. That is, in the moving object, a communication method may be implemented based on various methods, but the present disclosure is not limited to the above-described embodiments.

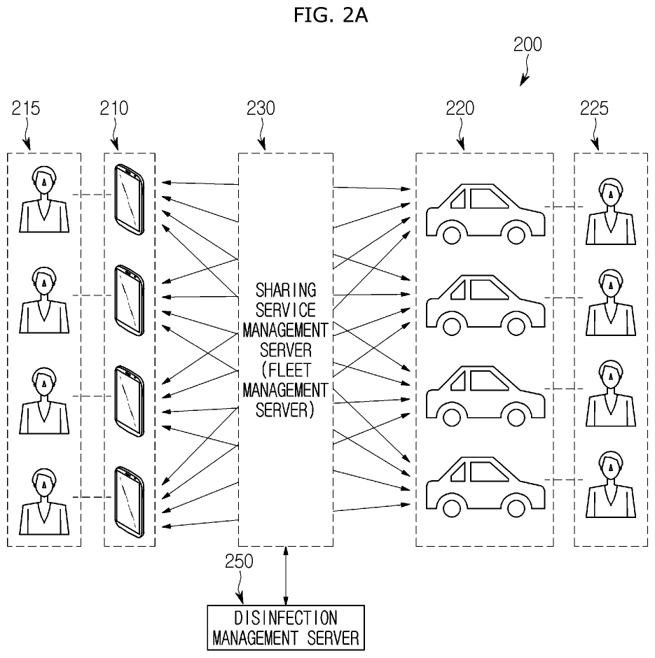

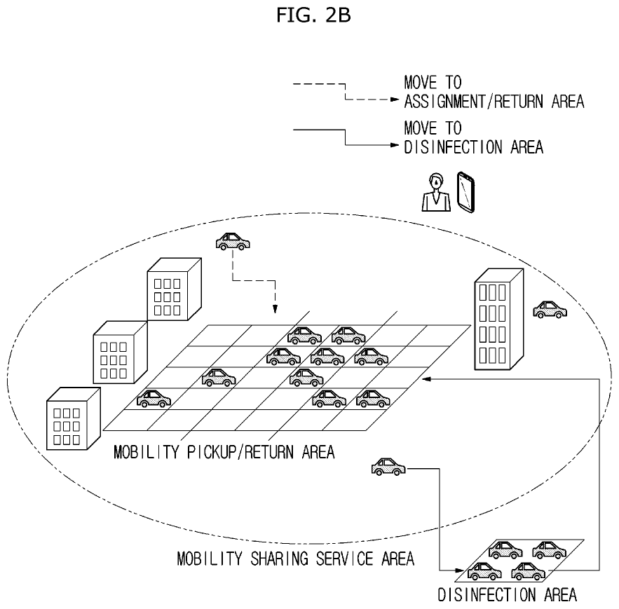

In addition, FIG. 2A is a view schematically illustrating a mobility sharing service system based on disinfection information according to an embodiment of the present disclosure.

In an embodiment of the present disclosure, a mobility sharing service system 200 may include a fleet management system and may include configuration and operation of a typical fleet management system.

The mobility sharing service system 200 may include a plurality of user terminals 210 and a plurality of mobilities 220.

For example, a user 215 who uses a mobility 220 may have a user terminal 210. In this case, the user 215 may use the mobility 220 based on the user terminal 210. For example, the user terminal 210 may be a smart device. As another example, the user terminal 210 may be a device mounted on a person. For example, the user terminal 210 may be a smart watch or other accessories. As another example, the user terminal 210 may include a SIM card. For example, the SIM card may be a portable device. As another example, the user terminal 210 may be a separate device for a fleet system. That is, the user terminal 210 may be a device for recognizing a mobility 220 and is not limited to the above-described embodiment.

Meanwhile, each owner 225 may have each of a plurality of mobilities 220. That is, there are owners 225 for the plurality of mobilities 220, which may be allowed to be used by another user 215 based on the mobility sharing service system. For example, owner's devices may correspond to the plurality of mobilities 220, respectively. That is, there is a device corresponding to an owner for each mobility 220.

For example, the mobility 220 may be a vehicle. In addition, for example, the mobility 220 may be an object moving in a given area, such as a rail. As another example, the mobility 220 may be a flying object such as a drone. That is, the mobility 220 may refer to a movable object and mean a mobility 220 shared based on a fleet system. In an embodiment of the present disclosure, although the mobility 220 is described as a vehicle in the following description for convenience of description, the present disclosure is not limited thereto and various types of mobilities are adaptively applicable.

The mobility sharing service system 200 may further include a sharing service management server 230 for providing a mobility sharing service, and the user terminal 210 and the mobility 220 may be connected to the sharing service management server 230 through a network.

The sharing service management server 230 may manage information for providing a mobility sharing service, for example, a list of mobilities, state information of a mobility, location information of a mobility, and rating information of a mobility, and may manage provision of a mobility sharing service based on the information. For example, the sharing service management server 230 may provide the list of mobilities managed in the system, and provide an environment in which the user 215 may select and use the mobility 220. In this case, the sharing service management server 230 may also provide information necessary to select the mobility 220, for example, the state information of the mobility, the location information of the mobility and the rating information of the mobility.

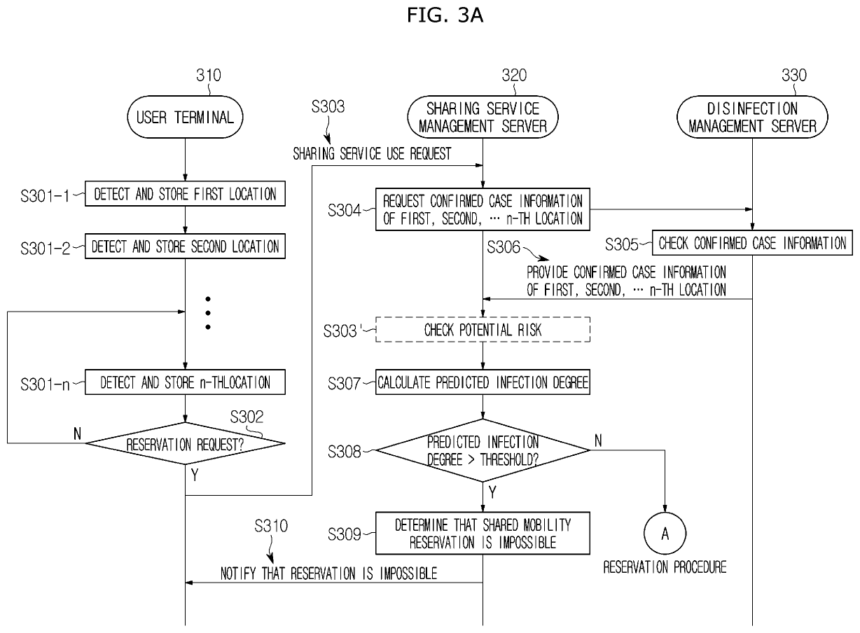

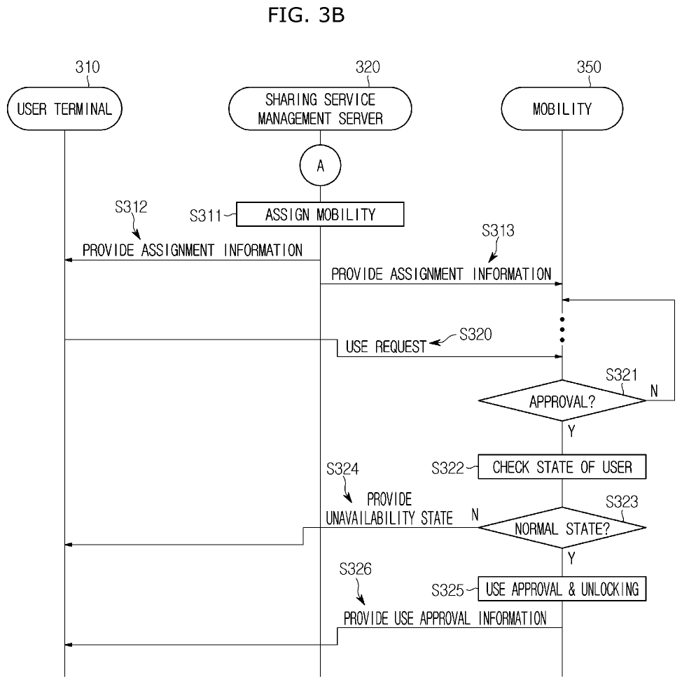

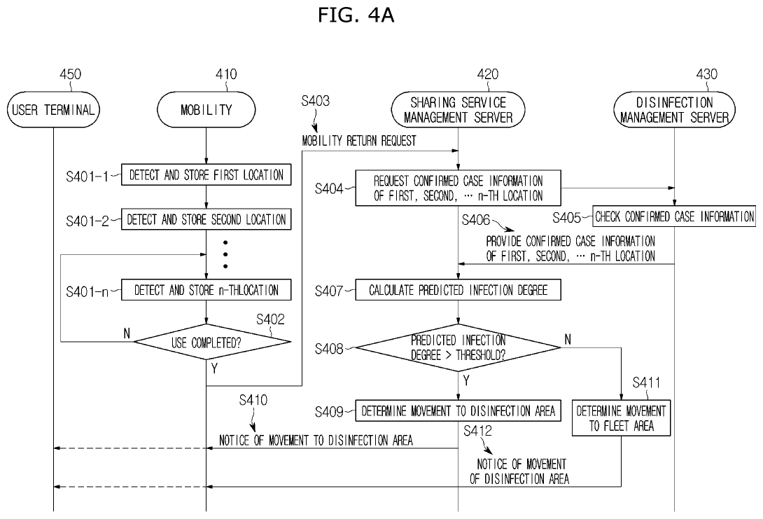

In particular, the sharing service management server 230 may receive disinfection information from a disinfection management server 250 and determine whether the user 215 uses the mobility based on the disinfection information or determine whether to reuse a mobility, use of which is completed. Operation of the sharing service management server 230 to perform the mobility sharing service based on the disinfection information will be described in detail below with reference to FIGS. 3, 4A and 4B.

Meanwhile, the disinfection management server 250 is a server device which stores and manages disinfection information and may store and manage disinfection information of a confirmed case of an infectious disease, for example. Here, the disinfection information may include personal information of the confirmed case, the type of the disease, and location information of the confirmed case according to time. Further, the disinfection management server 250 may store and manage the personal information of the confirmed case, the type of the disease, etc. based on the location information.

Furthermore, a server application (or program) for providing the mobility sharing service may be installed on the sharing service management server 230, and a client application (or program) for providing the mobility sharing service may be installed on the plurality of user terminals 210 and the plurality of mobilities 220. The server application (or program) and the client application (or program) for providing the mobility sharing service may be configured to interwork with each other, to request and receive information (or data) required to execute the application (or program) from each other, and to drive the application (or program) using the received information (or data). In addition, the server application (or program) and the client application (or program) for providing the mobility sharing service may be configured based on operation of performing the mobility sharing service described below.

FIG. 2B is a view schematically illustrating a mobility sharing service area and a disinfection area used in a mobility sharing service system based on disinfection information according to an embodiment of the present disclosure.

Referring to FIG. 2B, the mobility sharing service system based on the disinfection information may be configured to provide the mobility sharing service in a mobility sharing service area. For example, the mobility sharing service area may include an administrative district and a district set by a district unit plan. Accordingly, the mobility sharing service system based on the disinfection information may be configured such that the mobility may move in the mobility sharing service area. For example, the mobility sharing service system based on the disinfection information may set the mobility sharing service area and restrict driving of the mobility not to leave the corresponding area or manage rights to control the mobility. As another example, the mobility sharing service system based on the disinfection information may check whether the mobility approaches or departs from the boundary of the mobility sharing service area and induce entry into the mobility sharing service area using an alarm or a guide message. Although, in an embodiment of the present disclosure, the mobility sharing service area includes an administrative district and a district set by a district unit plan, the present disclosure is not limited thereto and various modifications are possible. As another example, the mobility sharing service area may include not only a physically set area but also an area where the mobility moves using the mobility sharing service.

Furthermore, the mobility sharing service area may include an area where the mobility is picked up and returned while using the service (hereinafter referred to as “mobility pickup/return area”). For example, the mobility pickup/return area may include a preset parking lot, an area provided for charging or refueling, and an area provided for car washing.

In particular, the mobility sharing service system based on the disinfection information may set and manage the disinfection area. For example, the mobility sharing service system based on the disinfection information may check a predicted infection degree and control or induce movement of the mobility to the disinfection area based on this (the predicted infection degree).

FIGS. 3A and 3B are signal flow diagrams illustrating operation of a mobility sharing service system based on disinfection information according to an embodiment of the present disclosure.

First, referring to FIG. 3A, a user terminal 310 may drive a client application (or program), request and receive location information of the user terminal from a location information processor (e.g., a GPS module) every predetermined time unit while the client application (or program) is driven, and periodically store the location information (S301-1, S301-2, S301-n) (where, n is a natural number). As another example, the user terminal 310 may check payment information of a user through a server for providing payment information (e.g., a card company server or a payment gateway server (e.g., a payment gateway (PG)), and check visit place information of the user based on the payment information of the user. The visit place information of the user may be temporarily stored for a predetermined time (e.g., 3 days) and may be used in step S303 below. Furthermore, the visit place information may include a time when the user visits a store and a detailed address of the visited store. Additionally, the user terminal 310 may check a user movement path based on the visit place information or check the movement scope of the user. The user movement path and the user movement scope may be checked based on information checked through a geomagnetic sensor, a location information processor (e.g., a GPS module) and a map application provided in the user terminal 310.

The user may input a mobility sharing service use request through the user terminal 310, and the user terminal 310 may check whether an event for the mobility sharing service use request has occurred in response thereto. For example, the event for the mobility sharing service use request may occur as a “menu (or UI) for a request to use a mobility sharing service” provided by the client application (or program) is selected.

The user terminal 310 may check that the event for the mobility sharing service use request has occurred, and transmit a sharing service use request to the sharing service management server 320 (S303). In addition, the sharing service use request may include user information and location information of the user terminal.

As the sharing service use request is received, the sharing service management server 320 may check the location of the user terminal included in the sharing service use request and request confirmed case information corresponding to the location of the user terminal from the disinfection management server 330 (S304).

The disinfection management server 330 may check the confirmed case information corresponding to the location of the user terminal and provide the checked information to the sharing service management server 320 (S305 and S306).

The sharing service management server 320 may receive the confirmed case information corresponding to the location of the user terminal and calculate a predicted infection degree based on the received information (S307). Here, the predicted infection degree may indicate in which degree the user may be infected. For example, the predicted infection degree may be in the form of numerical data. As another example, the predicted infection degree is data indicating the probability of infection stepwise and include, for example, a first step and a tenth step. The first step may be set to have a relatively lower probability of infection than the tenth step. Although the first step may be set to have a relatively lower probability of infection than the tenth step in the embodiment of the present disclosure, the present disclosure is not limited thereto and various modifications are possible. Furthermore, the predicted infection degree may be calculated by considering environmental factors such as population density, a rate of confirmed cases in an area, a rate of confirmed cases according to age and an increase in the number of confirmed cases. The environmental factors may be received from the disinfection management server 330 or may be received by the sharing service management server 320 from an external server and calculated. As another example, the predicted infection degree may be calculated by considering personal factors such as contact risk, user activity, an immunity index, and user information. As another example, the predicted infection degree may be calculated by considering a combination of the environmental factors and the personal factors.

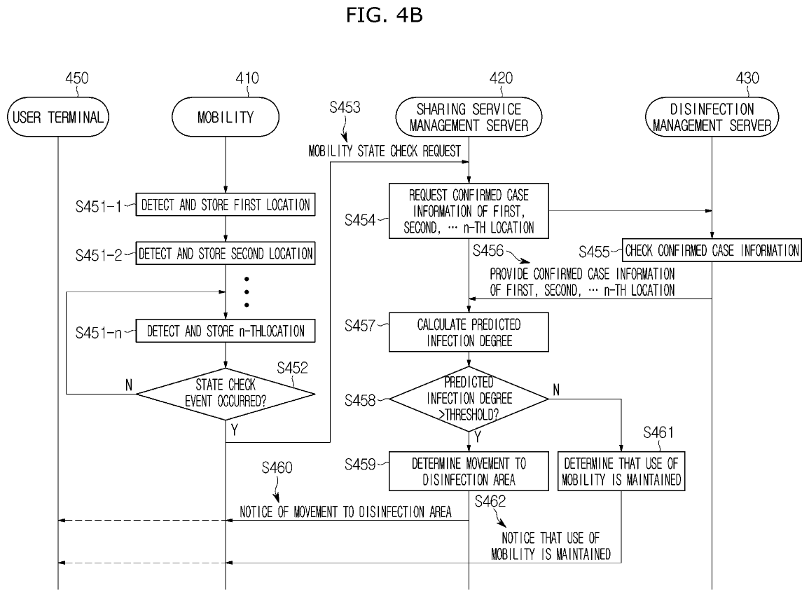

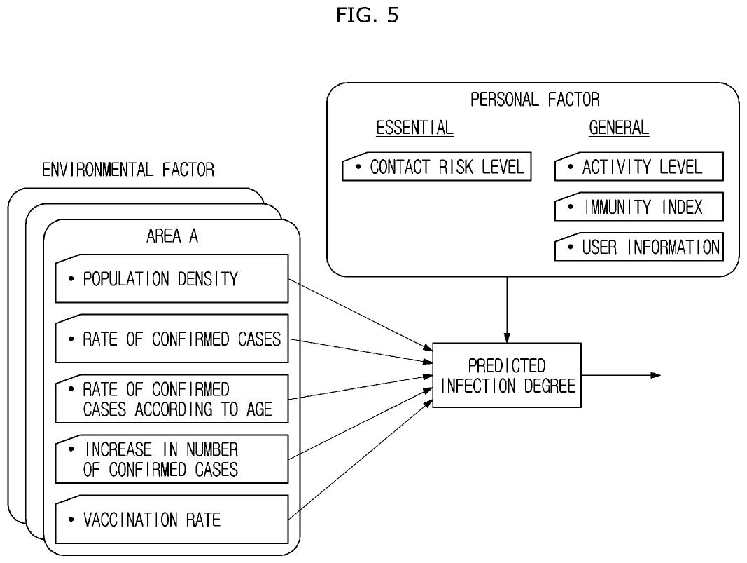

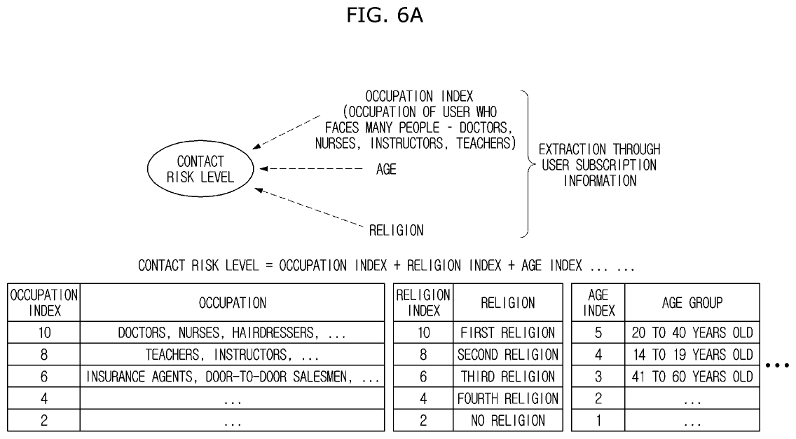

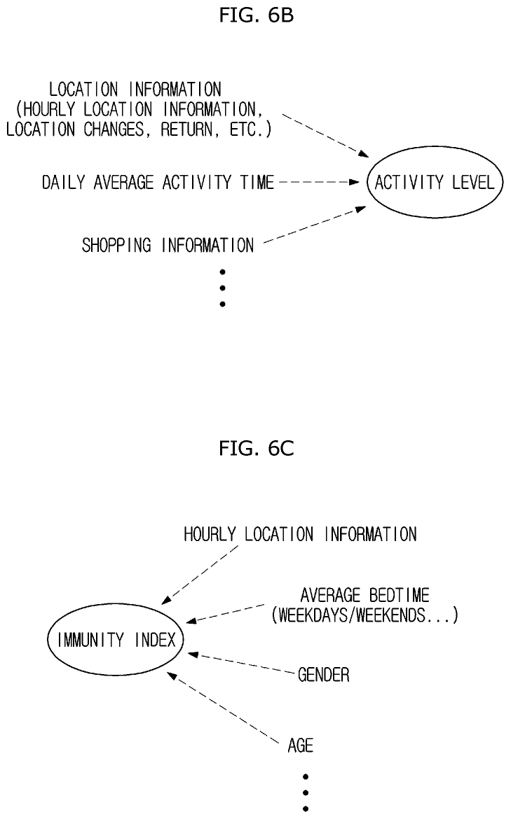

Furthermore, operation of calculating the predicted infection degree of the sharing service management server 320 will be described in detail with reference to FIGS. 5 to 6.

Meanwhile, the sharing service management server 320 may determine whether to use the sharing service by comparing the predicted infection degree with a predetermined threshold (S308). For example, the sharing service management server 320 may determine that the probability of infection of the user is relatively high and the shared mobility reservation is impossible (S309) when the predicted infection degree is relatively greater than the predetermined threshold (Y at S308), and notify the user terminal 310 of the user that the mobility sharing service is not available (S310), such that the mobility sharing service is not used.

In contrast, when the predicted infection degree is relatively less than the predetermined threshold (N at S308), the sharing service management server 320 may perform reservation operation of the mobility sharing service. For example, referring to FIG. 3B, the sharing service management server 320 may check user information and perform operation of assigning a mobility based on the user information (S311). In this case, the sharing service management server 320 may check mobility usage time information, departure point information, return point information, etc. The mobility usage time information, the departure point information, the return point information, etc. may be requested and received from the user terminal 310 while performing the operation of assigning the mobility. As another example, the mobility usage time information, the departure point information, the return point information, etc. may be provided to the sharing service management server 320 while transmitting the sharing service use request in S303. The operation of assigning the mobility of the sharing service management server 320 may be set based on a user's rating, the mobility usage time information, the departure point information and the return point information, and a detailed operation may be processed by a method provided by a general fleet service.

Thereafter, the sharing service management server 320 may provide assignment information including information on the assigned mobility to the user terminal 310 and the mobility 350 (S312 and S313). Here, the assignment information may include user information, mobility usage time, an identifier of the used mobility and approval key information.

The user may approach and use the mobility. In this case, the user may transmit use request information to the mobility 350 using the user terminal 310 (S320). Here, the use request information may include user information, mobility usage time, an identifier of the used mobility and approval key information. The mobility 350 may perform user approval by comparing the assignment information (the user information, the mobility usage time, the identifier of the used mobility, the approval key information, etc.) received in S313 with the use request information (the user information, the mobility usage time, the identifier of the used mobility and the approval key information) (S321). When user approval is completed, the mobility 350 may check user state information (S322). The user state information may include state information indicating the symptoms of an infectious disease, and may include, for example, a user's body temperature information. To this end, the mobility 350 may include an apparatus (e.g., an electronic thermometer, etc.) capable of checking the user's body temperature information, and the apparatus may provide the checked body temperature information to the mobility 350 through a wired/wireless communication network.

When the user state information indicates an abnormal state (No at S323), the mobility 350 may not unlock a locking device and may provide information indicating unavailability to the user terminal 310 (S324). In this case, the mobility 350 may directly transmit the information indicating unavailability to the user terminal 310 through the sharing service management server 320. As another example, the mobility 350 may directly transmit the information indicating unavailability to the user terminal 310.

In contrast, when the user state information indicates a normal state (Y at S323), the mobility 350 may approve use and unlock the locking device (S325). In addition, the mobility 350 may provide information indicating use approval to the user terminal 310 (S326).

Additionally, the sharing service management server 320 further may perform operation of checking a potential risk (S303′). For example, the sharing service management server 320 may check expected visit place information of the user. The expected visit place information may be checked based on the mobility sharing service use history of the user. Specifically, the sharing service management server 320 may store and manage waypoints and destinations when the user uses the mobility sharing service, analyze the waypoints and the destinations, and check main waypoints and main destinations. For example, the waypoints and the destinations may be checked through navigation destinations or waypoints and GPS information. As another example, the sharing service management server 320 may check payment information of the user through a server for providing payment information (e.g., a card company server, a payment gateway server (e.g., a payment gateway (PG)), and may check the main waypoints and the main destinations based on the payment information of the user. When the confirmed case information matches a main waypoint and a main destination through comparison, the sharing service management server 320 may provide information guiding the user to avoid the main waypoint and the main destination. The information guiding the avoidance of the main waypoint and the main destination may be provided through the mobility 350 or the user terminal 310.

Furthermore, the sharing service management server 320 may check whether the confirmed case information provided by a disinfection management server 330 matches the main waypoints and the main destinations through comparison. In this case, the sharing service management server 320 may provide the main waypoints and the main destinations to the disinfection management server 330 and request and receive the confirmed case information corresponding to the main waypoints and the main destinations.

Step S303′ is preferably performed after the sharing service use request operation S303 is performed. In addition, step S303′ is preferably performed before performing operation S307 of calculating the predicted infection degree. Meanwhile, as another example, the sharing service management server 320 may determine whether the mobility sharing service is available in consideration of the result of comparing the confirmed case information with the main waypoints and the main destinations. For example, when the user inputs the main waypoints and the main destinations as the destination or waypoint of the mobility sharing service, the sharing service management server 320 may determine that the mobility sharing service is unavailable. In addition, the sharing service management server 320 may notify that the mobility sharing service is unavailable. In this case, since the destination or waypoint of the mobility sharing service is required to be confirmed, step S303′ may be performed after confirmation of the destination or waypoint of the mobility sharing service is completed.

FIG. 4A is a signal flow diagram illustrating a mobility return operation of a mobility sharing service system based on disinfection information according to an embodiment of the present disclosure.

First, referring to FIG. 4A, while the user uses a mobility 410 through a mobility sharing service, the mobility 410 may drive a client application (or program), request and receive location information of the mobility from a location information processor (e.g., a GPS module) every predetermined time unit while driving the client application (or program) and periodically store location information (S401-1, S401-2 and S401-n) (where, n is a natural number). As another example, a user terminal 450 may check payment information of a user through a server for providing payment information (e.g., a card company server or a payment gateway server (e.g., a payment gateway (PG)), and check the visit place information of the user based on the payment information of the user. The visit place information of the user may be temporarily stored for a predetermined time (e.g., 3 days), and may be used in step S403 described below. Furthermore, the visit place information may include a time when the user visits a store and a detailed address of the visited store. Additionally, the user terminal 450 may check a user movement path based on the visit place information or check the movement scope of the user. The user movement path and the user movement scope may be checked based on information checked through a geomagnetic sensor, a location information processor (e.g., a GPS module) and a map application provided in the user terminal 450.

Although, in an embodiment of the present disclosure, the user terminal 450 checks the visit place information of the user through communication with a server for providing payment information (e.g., a card company server or a payment gateway server (e.g., a payment gateway (PG)), the present disclosure is not limited thereto and various modifications are possible. For example, the mobility 410 may check the visit place information of the user through communication with a server for providing payment information (e.g., a card company server or a payment gateway server (e.g., a payment gateway (PG)).

As described above, a sharing service management server 420 may provide destination information of the mobility sharing service to the mobility 410 in a process S313 of providing the assignment information, and the mobility 410 may generate and transmit a mobility return request to the sharing service management server 420 (S403), as the mobility enters an area close to the destination based on the location information and the destination information. For example, as the mobility 410 arrives at a location close to a predetermined distance from the destination, a mobility return request event may be generated and a mobility return request may be transmitted to the sharing service management server 420. In this case, the mobility return request may include user information, mobility information, location information of the mobility, etc.

As the mobility return request is received, the sharing service management server 420 may check the location information of the mobility included in the mobility return request, and request confirmed case information corresponding to the location of the mobility from a disinfection management server 430 (S404).

The disinfection management server 430 may check confirmed case information corresponding to the location of the mobility and provide the checked information to the sharing service management server 420 (S405, S406).

The sharing service management server 420 may receive the confirmed case information corresponding to the location of the mobility and calculate a predicted infection degree based on the received information. Here, the predicted infection degree may be calculated by considering environmental factors such as population density, a rate of confirmed cases in an area, a rate of confirmed cases according to age and an increase in the number of confirmed cases. The environmental factors may be received from the disinfection management server 430 or may be received by the sharing service management server 420 from an external server and calculated. As another example, the predicted infection degree may be calculated by considering personal factors such as contact risk, user activity, an immunity index, and user information. As another example, the predicted infection degree may be calculated by considering a combination of the environmental factors and the personal factors.

Furthermore, operation S407 of calculating the predicted infection degree of the sharing service management server 420 may be performed equally to operation S307 described above and will be described in detail with reference to FIGS. 5 and 6.

Meanwhile, the sharing service management server 420 may determine whether to continuously use the mobility for the sharing service or whether to move the mobility to a disinfection area to perform a disinfection process, by comparing the predicted infection degree with a predetermined threshold. For example, the sharing service management server 420 determines that the probability of infection of a subsequent user of the mobility is relatively high when the predicted infection degree indicates a relatively larger value than the predetermined threshold (Y at S408), and determines that the mobility 410 moves to the disinfection area (S409). In addition, the sharing service management server 420 may transmit, to the mobility 410, information indicating movement of the mobility 410 to the disinfection area (S410). For example, the sharing service management server 420 may set destination information of the mobility 410 to a disinfection area. Furthermore, the sharing service management server 420 may provide the user terminal 450 with information indicating movement of the mobility 410 to the disinfection area.

Meanwhile, when the predicted infection degree indicates a value equal to or relatively smaller than the predetermined threshold (N at S408), the sharing service management server 420 may determine that the mobility 410 moves to a fleet service area (S411). In addition, the sharing service management server 420 may provide information indicating movement to the fleet service area to the mobility 410 (S412). The sharing service management server 420 may provide the user terminal 450 with information indicating movement to the fleet service area.

Although, in an embodiment of the present disclosure, the predicted infection degree of the mobility is calculated in the mobility return process and operation of the mobility is controlled based on this, the present disclosure is not limited thereto and various modifications are possible. For example, the mobility sharing service system based on the disinfection information may calculate the predicted infection degree of the mobility every predetermined time unit and control operation of the mobility based on the predicted infection degree of the mobility when the user uses the mobility. Hereinafter, an operation of controlling operation of the mobility based on the predicted infection degree when the user uses the mobility will be described with reference to FIG. 4B.

FIG. 4B is a signal flow diagram illustrating a mobility control operation of a mobility sharing service system based on disinfection information according to an embodiment of the present disclosure.

First, referring to FIG. 4B, while the user uses the mobility 410 through the mobility sharing service, the mobility 410 may drive the client application (or program), request and receive location information of the mobility from a location information processor (e.g., a GPS module) every predetermined time unit while the client application (or program) is driven, and periodically store the location information (S451-1, S451-2, S451-n) (where, n is a natural number).

The mobility 410 may generate an event for checking the state of the mobility (hereinafter referred to as “state check event”) every predetermined time unit (S452) and transmit a mobility state check request to the sharing service management server 420 whenever the state check event is generated (S453). In this case, the mobility state check request may include user information, mobility information and location information of the mobility.

As the mobility state check request is received, the sharing service management server 420 may check the location of the mobility included in the mobility state check request and request confirmed case information corresponding to the location of the mobility from the disinfection management server 430 (S454).

The disinfection management server 430 may check the confirmed case information corresponding to the location of the mobility and provide the checked information to the sharing service management server 420 (S455 and S456).

The sharing service management server 420 may receive the confirmed case information corresponding to the location of the mobility and calculate a predicted infection degree based on the received information. Here, the predicted infection degree may be calculated by considering environmental factors such as population density, a rate of confirmed cases in an area, a rate of confirmed cases according to age and an increase in the number of confirmed cases. The environmental factors may be received from the disinfection management server 430 or may be received by the sharing service management server 420 from an external server and calculated. As another example, the predicted infection degree may be calculated by considering personal factors such as contact risk, user activity, an immunity index, and user information. As another example, the predicted infection degree may be calculated by considering a combination of the environmental factors and the personal factors.

Furthermore, operation S457 of calculating the predicted infection degree of the sharing service management server 420 may be performed equally to operation S307 described above and will be described in detail with reference to FIGS. 5 and 6.

Meanwhile, the sharing service management server 420 may determine whether to maintain use of the mobility for the sharing service or whether to move the mobility to a disinfection area to perform a disinfection process, by comparing the predicted infection degree with a predetermined threshold (S458). For example, the sharing service management server 420 determines that the probability of infection of a user of the mobility is relatively high when the predicted infection degree indicates a relatively larger value than the predetermined threshold (Y at S458), and determines that the mobility 410 moves to the disinfection area (S459). In addition, the sharing service management server 420 may transmit, to the mobility 410, information indicating movement of the mobility 410 to the disinfection area (SS460). For example, the sharing service management server 420 may set destination information of the mobility 410 to a disinfection area. Furthermore, the sharing service management server 420 may provide the user terminal 450 with information indicating movement of the mobility 410 to the disinfection area.

Meanwhile, when the predicted infection degree indicates a value equal to or relatively smaller than the predetermined threshold (N at S458), the sharing service management server 420 may determine that use of the mobility 410 is maintained (S461). In addition, the sharing service management server 420 may provide the mobility 410 with information indicating that use of the mobility is maintained (S462).

FIG. 5 is a diagram illustrating a predicted infection level calculation operation used in a mobility sharing service system based on disinfection information according to an embodiment of the present disclosure.

Referring to FIG. 5, in an embodiment of the present disclosure, a sharing service management server may calculate a predicted infection degree, and may calculate the predicted infection degree using at least one of the environmental factors or personal factors.

The environmental factors may include at least one of population density for a specific area, a rate of confirmed cases, a rate of confirmed cases according to age, an increase in the number of confirmed cases or a vaccination rate. Here, the population density may be information indicating the population density calculated for each administrative district unit of the corresponding area. The rate of confirmed cases may include a value calculated by dividing the number of confirmed cases in the corresponding area (administrative district or predetermined district range) by the total population. The rate of confirmed cases according to age may include information obtained by dividing the above-described rate of confirmed cases in predetermined age units (e.g., 0 to 10, 11 to 20, 21 to 30, . . . ). The increase in the number of confirmed cases may include information indicating an increase/decrease in the number of confirmed cases based on the number of confirmed cases accumulated in predetermined time units (e.g., 1 day, 3 days, 1 week, 10 days, . . . ). Furthermore, the vaccination rate may include a total vaccination rate, an age-specific vaccination rate.

The personal factors may include a contact risk level as an essential factor. The contact risk level is calculated based on user information and may be set such that a user having relatively many interpersonal contacts has a high value. An operation of calculating the contact risk degree will be described in detail with reference to FIG. 6A.

In addition, the personal factors may further include at least one of a user's activity level, a user's immunity index or user information as general factors. Furthermore, an operation of calculating the user's activity level will be described in detail with reference to FIG. 6B and operation of calculating the user's immunity index will be described in detail with reference to FIG. 6C.

The sharing service management server may preferentially check an essential element to check whether the essential element exceeds a predetermined threshold and calculate the predicted infection degree by applying a general element as the essential element exceeds the predetermined threshold. In this case, the sharing service management server may calculate the predicted infection degree by applying a predetermined weight to the general element. In contrast, as the essential element represents a value equal to or relatively smaller than a predetermined threshold, the sharing service management server may calculate the predicted infection degree using only the essential element.

FIG. 6A is a diagram illustrating a contact risk level used in a mobility sharing service system based on disinfection information according to an embodiment of the present disclosure.

Referring to FIG. 6A, the contact risk level may be calculated by combining at least one of an occupation index, a religion index and an age index. Since the contact risk level has a high value for a user who has relatively many interpersonal contacts, a relatively high occupation index may be given to an occupation of a user who faces relatively many people in a close place. For example, a highest occupation index (e.g., 10) may be given to doctors, nurses and hairdressers, a second highest occupation index (e.g., 8) may be given to teachers or instructors, and a third highest occupation index (e.g., 6) may be given to insurance agents and door-to-door salesmen. In addition, the religion index may be calculated in consideration of the religious information of the user. For example, a relatively high religion index may be given to a religion in which religious activities are performed in a space where a relatively large number of people are densely populated. For example, a highest religion index (e.g., 10) may be given to a first religion, a second highest religion index (e.g., 8) may be given to a second religion, a third highest religion index (e.g., 6) may be given to a third religion, a fourth highest religion index (e.g., 4) may be given to a fourth religion, and a lowest religion index (e.g., 2) may be given to no religion. Furthermore, since the contact risk level has a high value for a user who has relatively many interpersonal contacts, a relatively high age index may be given to an age group of a user who faces relatively many people in a close place. For example, a highest age index (e.g., 5) may be given to an age group of 20 to 40 years old, a second highest age index (e.g., 4) may be given to an age group of 14 to 19 years old, and a third highest age index (e.g., 3) may be given to an age group of 41 to 60 years old.

Although, in an embodiment of the present disclosure, the occupation index, the religion index and the age index are described as information included in the contact risk level, the present disclosure is not limited thereto and various modifications are possible within the range capable of giving high contact risk levels to users who have relatively many interpersonal contacts.

FIG. 6B is a diagram illustrating a user's activity level used in a mobility sharing service system based on disinfection information according to an embodiment of the present disclosure.

The user's activity level is information indicating how actively a user moves and may be calculated based on the location of the user, from which the user's activity may be predicted. For example, the user's activity level may be calculated based on the hourly location of the user, the number of times of changing the location of the user, and a daily average activity time. As another example, the user's activity level may be calculated using shopping information of the user. For example, the sharing service management server may check payment information of the user through a server for providing payment information (e.g., a card company server or a payment gateway server (e.g., a payment gateway (PG)), and numerically calculate how actively the user moves based on the payment information of the user.

FIG. 6C is a diagram illustrating a user's immunity index used in a mobility sharing service system based on disinfection information according to an embodiment of the present disclosure.

The user's immunity index may indicate information numerically indicating a user's ability to defend against infectious diseases and may be calculated based on a usage pattern of a user terminal of the user or the location information of the user terminal. For example, whether the user terminal is used or not may be checked based on touch input, motion information and application operation, and a bedtime may be predicted based on this information. For example, the rest time, exercise time, etc. of the user may be predicted based on the location information of the user terminal. Based on this, the sharing service management server may check a bedtime, a rest time and an exercise time using the user terminal and calculate the immunity index of the user based on this information. Furthermore, the sharing service management server may calculate the immunity index by further considering user information. For example, since the immunity may vary according to the age or gender of the user, the sharing service management server may calculate the immunity index by further checking and applying user information such as the age or gender of the user.

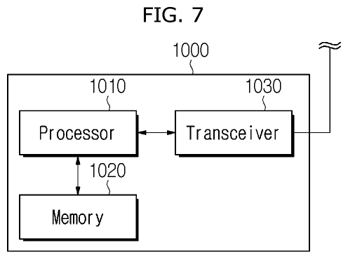

FIG. 7 is a diagram illustrating the configuration of an apparatus according to an embodiment of the present disclosure.

Referring to FIG. 7, the apparatus may include at least one of the above-described moving object, device, server or RSU. That is, the apparatus may perform communication with another device and may be linked to another device, and is not limited to the above-described embodiment. For example, the apparatus 1000 may include at least one of a processor low, a memory 1020 or a transceiver 1030 for the above-described operation. That is, the apparatus may include components necessary to perform communication with another device. In addition, for example, the apparatus may include components other than the above-described components. That is, the apparatus includes the above-described components to perform communication with another device, but is not limited thereto, and may operate based on the above description.

While the exemplary methods of embodiments of the present disclosure described above are represented as a series of operations for clarity of description, it is not intended to limit the order in which the steps are performed, and the steps may be performed simultaneously or in different order as necessary. In order to implement the methods according to embodiments of the present disclosure, the described steps may further include other steps, may include remaining steps except for some of the steps, or may include other additional steps except for some of the steps.

The various embodiments of the present disclosure are not a list of all possible combinations and are intended to describe representative aspects of the present disclosure, and the matters described in the various embodiments may be applied independently or in combination of two or more.

In addition, various embodiments of the present disclosure may be implemented in hardware, firmware, software, or a combination thereof. In the case of implementing embodiments of the present invention by hardware, embodiments of the present disclosure can be implemented with application specific integrated circuits (ASICs), digital signal processors (DSPs), digital signal processing devices (DSPDs), programmable logic devices (PLDs), field programmable gate arrays (FPGAs), general processors, controllers, microcontrollers, microprocessors, etc.

The scope of the disclosure includes software or machine-executable commands (e.g., an operating system, an application, firmware, a program, etc.) for enabling operations according to the methods of various embodiments to be executed on an apparatus or a computer, a non-transitory computer-readable medium having such software or commands stored thereon and executable on the apparatus or the computer.

What is claimed is:

1. A method of providing a mobility sharing service based on disinfection information, the method comprising:
receiving, at a mobility sharing management server, a mobility sharing service request from a user terminal;
requesting, by the mobility sharing management server, disinfection information from a disinfection management server based on location information of the user terminal;
receiving, by the mobility sharing management server, the disinfection information from the disinfection management server and calculating a predicted infection degree based on the disinfection information; and
determining whether to provide the mobility sharing service to a user corresponding to the user terminal based on the predicted infection degree.

2. The method of claim 1, wherein calculating the predicted infection degree comprises:
checking an environment factor corresponding to a mobility sharing service area or a personal factor corresponding to the user; and
calculating the predicted infection degree based on the environment factor or the personal factor.

3. The method of claim 2, wherein the environment factor comprises information on population density of the mobility sharing service area, information on a rate of confirmed cases in an area, information on a rate of confirmed cases according to age, or information on an increase in a number of confirmed cases.

4. The method of claim 2, wherein checking the personal factor comprises:
checking an essential element included in the personal factor; and
checking whether the essential element exceeds a predetermined threshold.

5. The method of claim 4, wherein checking the personal factor comprises checking a general element included in the personal factor in response to the essential element exceeding the predetermined threshold.

6. The method of claim 5, wherein the general element comprises an activity level of the user numerically indicating an activity tendency of the user or an immunity index of the user numerically indicating an ability to defend against a disease.

7. The method of claim 4, wherein checking the personal factor comprises:
checking the essential element or a general element included in the personal factor; and
determining the personal factor using the checked essential element or general element.

8. The method of claim 4, wherein the essential element comprises contact risk information numerically indicating an interpersonal contact tendency of the user.

9. The method of claim 1, further comprising:
transmitting, by a mobility, a mobility return request to the mobility sharing management server;
requesting, by the mobility sharing management server, disinfection information from the disinfection management server based on location information of the mobility;
receiving, by the mobility sharing management server, the disinfection information from the disinfection management server, and calculating the predicted infection degree of the mobility based on the disinfection information; and
determining whether to reshare the mobility using the predicted infection degree of the mobility.

10. The method of claim 9, wherein determining whether to reshare the mobility comprises determining whether to reshare the mobility by comparing the predicted infection degree of the mobility with a predetermined threshold.

11. The method of claim 10, wherein determining whether to reshare the mobility comprises determining that a subsequent user reshares the mobility in response to the predicted infection degree of the mobility being equal to or relatively smaller than the predetermined threshold.

12. The method of claim 11, wherein the method further comprises determining a destination of the mobility to be a return area.

13. The method of claim 10, wherein determining whether to reshare the mobility comprises determining a disinfection process for the mobility in response to the predicted infection degree of the mobility exceeding the predetermined threshold.

14. The method of claim 13, wherein determining the disinfection process for the mobility comprises determining a destination of the mobility to be a disinfection area.

15. The method of claim 9, wherein calculating the predicted infection degree of the mobility comprises:
checking environment factors corresponding to the location information of the mobility; and
calculating the predicted infection degree of the mobility based on the environment factors.

16. The method of claim 1, further comprising:
transmitting, by a mobility, a mobility state check request to the mobility sharing management server;
requesting, by the mobility sharing management server, disinfection information from the disinfection management server based on location information of the mobility;
receiving, by the mobility sharing management server, the disinfection information from the disinfection management server, and calculating the predicted infection degree of the mobility based on the disinfection information; and
determining whether to maintain use of the mobility.

17. The method of claim 16, wherein determining whether to maintain use of the mobility comprises determining whether to maintain use of the mobility by comparing the predicted infection degree of the mobility with a predetermined threshold.

18. The method of claim 17, wherein determining whether to maintain use of the mobility comprises determining a disinfection process of the mobility in response to the predicted infection degree of the mobility exceeding the predetermined threshold.

19. A mobility for use in a mobility sharing service, the mobility comprising:
a transceiver configured to transmit and receive a signal; and
a processor configured to:
control the transceiver;
approve use of the mobility sharing service by a user based on assignment information received from a mobility sharing management server and use request information received from a user terminal corresponding to the user;
check location information of the mobility during use of the mobility sharing service by the user; and
receive a determination as to whether to reshare the mobility from the mobility sharing management server.

20. A server apparatus for use in a mobility sharing service, the server apparatus comprising:
a transceiver configured to transmit and receive a signal; and
a processor configured to:
control the transceiver,
receive a mobility sharing service request from a user terminal;
request disinfection information from a disinfection management server based on location information of the user terminal;
receive the disinfection information from the disinfection management server and calculate a predicted infection degree based on the disinfection information; and
determine whether to provide the mobility sharing service to a user corresponding to the user terminal.
